(19) United States
(12) Patent Application Publication
(10) Pub. No.: US 2014/0377186 A1
(43) Pub. Date: Dec. 25, 2014

(54) MICROBUBBLE ULTRASOUND CONTRAST AGENT FOR EXTERNAL USE

(71) Applicants: National Defense Medical Center, Taipei City (TW); National Taiwan University of Science and Technology, Taipei (TW)

(72) Inventors: Ai-Ho Liao, Taipei City (TW); Chih-Hung Wang, New Taipei City (TW)

(73) Assignees: National Defense Medical Center, Taipei City (TW); National Taiwan University of Science and Technology, Taipei (TW)

(21) Appl. No.: 13/961,903

(22) Filed: Aug. 8, 2013

(30) Foreign Application Priority Data
Jun. 25, 2013 (TW) 102122588

(51) Int. Cl. A61K 49/18

(52) U.S. Cl. CPC A61K 49/1818
USPC 424/9.51; 424/9.52

(57) ABSTRACT

A microbubble ultrasound contrast agent for external use is provided. The microbubble ultrasound contrast agent applied externally can safely and efficiently enhance the permeation and absorption of the drug or small molecules in the local region of the body surface.

### CROSS-REFERENCE TO RELATED APPLICATION

This application claims the priority benefit of Taiwan application serial no. 102122588, filed on Jun. 25, 2013. The entirety of the above-mentioned patent application is hereby incorporated by reference herein and made a part of this specification.

### BACKGROUND

1. Technical Field

The present invention relates to a biomedical agent. Particularly, the present invention relates to an ultrasound microbubble contrast agent for external uses.

2. Related Art

For decades, ultrasound has been one of the most important tools in the medical or therapeutic field as it is an accurate, inexpensive and easily operated tool with no ionizing radiation. For the ultrasonic technology, the microbubble ultrasound contrast agent is applied intravascularly and the tiny bubbles of the microbubble ultrasound contrast agent in the blood vessel are excited by ultrasonic energy to generate harmonic resonance, which enhances the received ultrasound images. The application of the microbubble ultrasound contrast agents may help increase the contrast resolution and sensitivity of high-frequency ultrasound imaging. However, as the conventional microbubble ultrasound contrast agent is injected into the blood vessel or into the living body, an overall risk of applying the conventional microbubble ultrasound contrast agent is somehow higher, which is detrimental for medical or research applications.

### SUMMARY

The present invention provides an external type microbubble ultrasound contrast agent of topical uses. The microbubble ultrasound contrast agent may be applied to a topical region of the body surface of the living body by coating, instead of using injection. The external type microbubble ultrasound contrast agent may employ a medium, either aqueous or a gel form, and contain microbubbles of a specific particle size and at a specific concentration. The material of the microbubbles may be albumin, liposomes, polymers, copolymers or mixtures of the aforementioned material or a combination of those above. The external type microbubble ultrasound contrast agent may be applied in conjunction with the application of mechanical oscillation waves. Through a series of swelling and shrinking processes induced by the oscillation energy of the mechanical oscillation waves, the microbubbles burst or destructed and the generated energy and shock: waves lead to minor damages of cells or tissues, which further strengthen the absorption of applied chemicals or small molecules. The commonly used energy source of the mechanical oscillation waves may be a source of an optical energy or acoustic energy, such as an ultrasound source or a laser source. The external type microbubble ultrasound contrast agent of the present invention, suitable for applying onto a local region of the body surface of the living body, may be used in combination with the mechanical wave(s) generated by the mechanical oscillating energy source to cause the microbubbles in the external type microbubble ultrasound contrast agent bursting to produce energy and shock waves. The energy and the shock waves from microbubble bursting cause minor and reversible damages on the contact area of the skin surface or mucous membrane, thereby increasing the percutaneous absorption of chemicals or small molecules. The microbubble ultrasound contrast agent may be widely used in medical or beauty fields, to help strengthen the absorption of painkillers after surgery or the absorption of beauty care ingredients.

The present invention provides an external type microbubble ultrasound contrast agent including a medium and a plurality of microbubbles dispersed in the medium. The medium is in a form of an aqueous solution or a gel form and a concentration of the microbubbles ranges from 1×10⁹ to 2×10⁹ particles/ml.

According to embodiments of the present invention, the material of the microbubbles is selected from albumin, polymers, liposomes, copolymers or mixtures thereof or a combination of thereof, and the medium is selected from an isotonic saline solution, an agar gel, an aloe gel, a topical gel or a combination of thereof.

According to embodiments of the present invention, the medium is a gel form medium and a content of the gel form medium is 0˜0.2 percentages by weight of a total weight of the microbubble ultrasound contrast agent.

According to embodiments of the present invention, a particle size of the microbubbles ranges from 0.5 micrometers to 2.5 micrometers.

According to embodiments of the present invention, the microbubble ultrasound contrast agent farther includes a chemical or small molecules, and the chemical or the small molecules are percutaneously absorbed by a biological body.

The present invention also provides a method for enhancing percutaneous absorption of a chemical or small molecues through a topical region of a biological body surface. A microbubble ultrasound contrast agent is applied to the topical region of the biological body surface. The microbubble ultrasound contrast agent comprises a medium and a plurality of microbubbles dispersed in the medium, the medium is in a form of an aqueous solution or a gel form, and a material of the microbubbles is selected from albumin, polymers, liposomes, copolymers or mixtures thereof or a combination of thereof. Also, a chemical or small molecules are applied to the topical region. Then, a mechanical oscillation wave source is applied to the topical region to be in direct contact with the topical region applied with the microbubble ultrasound contrast agent and the chemical or the small molecules. Through mechanical waves generated by the mechanical oscillating energy source acting on the microbubbles, the percutaneous absorption of the chemical or the small molecules is enhanced.

According to embodiments of the present invention, a concentration of the microbubbles ranges from 2×10⁶ to 2×10⁸ particles/ml, relative to the total volume of the microbubble ultrasound contrast agent and the chemical or the small molecules.

According to embodiments of the present invention, using the chemical or the small molecules as a diluent, the microbubble ultrasound contrast agent is diluted 2-1000 times.

According to embodiments of the present invention, the steps of applying the microbubble ultrasound contrast agent and applying the chemical or the small molecules are performed individually and not at the same time.

According to embodiments of the present invention, a particle size of the microbubbles ranges from 0.5 micrometers to 2.5 micrometers.

According to embodiments of the present invention, the mechanical oscillation wave source includes an ultrasound source and/or a laser source.

Based on the above, the present invention provides an external type ultrasound microbubble contrast agent(s), which can safely and effectively enhance the absorption or penetration of the chemical or small molecules at the topical region and avoid the risk of allergies by injecting the contrast agent into the body.

In order to make the aforementioned and other features and advantages of the disclosure comprehensible, several exemplary embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a flow chart illustrating the application of the ultrasound microbubble ultrasound contrast agent together with the treatment of ultrasound according to one embodiment of the present invention.

FIG. 2 is a schematic view of a penetration-through experimental system with the tissue simulator according to one embodiment of the present invention.

FIG. 3A shows the penetration depth of the agar stimulator in the penetration-through experiments according to one embodiment of the present invention.

FIG. 3B is a quantitative diagram showing the relationship of the penetration depth of the agar stimulator in the penetration-through experiments and the standing time according to one embodiment of the present invention.

FIG. 4A is a 100-fold magnification showing the percutaneous penetration depth of the penetration-through experiments.

FIG. 4B is a 400-fold magnification showing the percutaneous penetration depth of the penetration-through experiments.

FIG. 5 shows the results of the delivery efficiency using different administration approaches of the microbubble contrast agent in the inner ear treatment experiments.

FIGS. 6A˜6F show the delivery results of the green dye indicator entering into the round window membrane cells of the inner ear under different administration approaches.

FIGS. 7A-7B show the results of the auditory brainstem response tests of the animals following the animal tests.

### DETAILED DESCRIPTION OF DISCLOSED EMBODIMENTS

The microbubble ultrasound contrast agent of the present invention may be of an aqueous solution or a gel form and contain the microbubbles of a specific particle size and a specific concentration. According to the material of the microbubbles contained therein, the microbubble ultrasound contrast agent can be divided roughly into three categories: albumin microbubbles, liposome microbubbles or polymer microbubbles. The microbubbles contained in the microbubble ultrasound contrast agent have stable shells and may be used to enhance the scattering signals of reflected ultrasound. Under various ultrasound energy intensities, using the microbubble ultrasound contrast agent can increase the penetration depth (i.e. absorption efficiency) and/or the amount of penetration (i.e. absorption) of the chemicals or small molecules at the applied area.

Taking the lipid microbubble ultrasound contrast agent as an example, under the action of very low sound field energy of the mechanical index (MI) less than 0.05˜0.1, the microbubble ultrasound contrast agent oscillate linearly and symmetrically. When the mechanical index is raised to 0.1˜0.3, the microbubble ultrasound contrast agent is being squeezed more than being relaxed. At this time, although the microbubble ultrasound contrast agent does not have considerable cavitation, the microbubble ultrasound contrast agent has significant nonlinear response and the signal spectrum has obvious harmonic components. Harmonic imaging can effectively increase the scattering ratios of the bubbles to the tissues. However, in the case of high sound pressure (mechanical index greater than 0.3˜0.6), the microbubble ultrasound contrast agent may endure big squeezes and relaxations, leading to the bursting of the microbubbles in the microbubble ultrasound contrast agent into pieces and then linear scattering and cavitation. Shock waves generated by cavitation can cause membrane perturbation and increase its permeability. According to the studies, under the high sound field, cavitation of the microbubble ultrasound contrast agent is used to augment microvascular leakage, inflammatory cell infiltrations, hemolysis or even capillary ruptures and so on.

The present invention provides an external type microbubble ultrasound contrast agent, and the microbubble ultrasound contrast agent may be applied to a topical region of the body surface of the living body (i.e. external) by coating, painting or spraying. The external use microbubble ultrasound contrast agent(s) can enhance the absorption efficacy of chemicals or small molecules that are mixed with the microbubble ultrasound contrast agent(s) by the topical region of the living body. Compared to the previously used microbubble ultrasound contrast agent that is injected into the living body's circulatory systems, the microbubble ultrasound contrast agent of the present invention is designed to be the medium disposed between the ultrasound probe and the action site (a local region of the biological body surface, such as, face, ear cavity or joints, etc.). That is, the microbubbles exist stably in the microbubble ultrasound contrast agent and the microbubbles are in direct contact with the ultrasound probe to induce cavitation under the ultrasound energy, thereby strengthening the absorption and utilization of chemicals or small molecules applied to shallow parts of the body surface. Further, since the chemicals or small molecules mixed with the microbubble ultrasound contrast agents of the present invention are not enveloped within the microbubbles, the chemicals or small molecules may be used with the microbubble ultrasound contrast agents of the present invention separately or in combination. In other words, these chemicals, small molecules may be applied or coated to the outside surface of the living body in different orders.

The microbubble ultrasound contrast agents of the present invention may be designed to adjust the microbubble concentration and/or medium tension to make the formulation of the microbubble ultrasound contrast agent appropriate for being in direct contact with the ultrasound probe. The medium of the microbubble ultrasound contrast agents may be an aqueous medium or in a gel form, and the medium still has effective acoustic transfer properties with a specific concentration of microbubbles. The material of the microbubbles in the microbubble ultrasound contrast agent may be albumin, liposomes, polymers, copolymers or mixtures of the foregoing material(s), or a combination of the above.

The present invention also describes the application of the ultrasound microbubble composition for external uses, applied to a local region of the body surface to promote the penetration efficacy of chemicals or small molecules through the skin or mucous membranes of the local region, so as to strengthen the absorption of those chemicals or small molecules. Such external use ultrasound microbubble composition includes at least one medium and a plurality of microbubbles dispersed in the medium. The medium may be in the form of an aqueous solution or a colloid suspension, and the material of the microbubbles may be selected from albumin, polymers, liposomes, copolymers or mixtures of the aforementioned material(s), or a combination of the above. When used, the topical ultrasound microbubble composition may be diluted 2-1000 times, from the original prior concentration of 1×10⁹˜2×10⁹ particle/ml to the concentration range of 2×10⁶˜2×10⁸ particles/ml by adding the medium.

The following examples are based on albumin microbubble ultrasound contrast agent(s), for example, but the microbubble ultrasound contrast agent of the present invention is not limited to the content of the following Examples.

### EXAMPLES

Preparation steps of topical microbubble contrast agent:

Method one: the preparation of aqueous microbubble ultrasound contrast agent(s).

The isotonic saline solution and 1.2 wt % of human serum albumin (HSA, purchased from Octapharma, Vienna, Austria) were uniformly mixed into 10 ml of the solution, filled with C₃F₈ gas, and oscillated for two minutes using the ultrasonic cell processor to prepare the microbubble ultrasound contrast agent. The microbubble ultrasound contrast agent contains microbubbles, formed in the oscillation process, with C₃F₈ gas sealed by albumin shells. After the oscillation was complete, the microbubble ultrasound contrast agent was dispensed into microcentrifuge tubes, placed in micro-centrifuge for separation (speed: 1200 rpm (128.7 g), time: 2 minutes), extract subnatant and add the appropriate amount of saline for storage at 4° C. refrigerator. For the contrast agent(s) used in this experiment, a concentration of the microbubbles is about 2×10⁹ particles/ml and the particle size distribution of the microbubbles is about 0.5˜2.5 μm.

Component A: using the isotonic saline solution as the medium to adjust concentrations of the microbubbles for the various commercial lipid-shell microbubbles (including phospholipid microbubbles SonoVue® (purchased from Bracco Diagnostics, Milan, Italy) or Targestar (purchased from Targeson, La Jolla, Calif.) or the prepared albumin-shell microbubble ultrasound contrast agent as mentioned above, to the concentrations of 1×10⁹˜2×10⁹ particles/ml (the microbubble liquid).

Component B: chemical, biological and other small molecules or drugs to be used together are prepared. The substance to be used together should be formulated in the desired state of an aqueous solution, an emulsion or a gel, so that the substance is isotonic with human cells with a pH=7.4. For example, chemical, biological or small molecule drugs may be painkillers (such as diclofenac), arbutin, vitamin C phosphate magnesium salt, whitening ingredients (such as nonapeptide-1), gentamycin or glucocorticoid and other applicable substances.

Component C: Component B is used a diluent to dilute Component A 2˜1000 times and the composition obtained after dilution is applied to the surface of the body. Most preferably, Component B is used to dilute Component A 2˜40 times; more preferably, Component B is used to dilute Component A 30˜150 times. Also, Component B is used to dilute Component A 100˜1000 times. According to experimental results, the 10-fold dilution was the best dilution solution applied to the skin surface. Other dilution ratios are effective, and the dilution ratios should be adjusted depending on the application region. In general, in the topical microbubble contrast agent, the concentration of microbubbles ranges preferably from about 2×10⁶˜2×10⁸ particles/ml.

In general, the ultrasonic probe directly applied on the outer surface of the living body is in direct contact with Component C for local application of ultrasound with the power of 0.1˜5 W/cm² and the mechanical index (MI) <1.9. In addition, the ultrasonic energy applied with Component C may be replaced by other sources capable of generating the mechanical oscillation energy or may be used in combination with other devices. For example, the therapeutic laser beams may be applied to the local region with Component C. The mechanical oscillation means functioned with Component C and the corresponding replacements may be easily conceived by the skilled persons, and examples herein are not used to limit the applied energy sources.

Method Two: the preparation of colloidal microbubble ultrasound contrast agent(s):

The isotonic saline solution is used to prepare 0.2 wt % or less of the agar gel, aloe vera gel, or other topical gel.

Component D: The topical gel as described above is used as the medium, and the microbubble ultrasound contrast agent and 0.2 wt % or less (for example, 0.1 wt % or 0.15 wt %) of the agar gel, aloe (vera) gel, or other topical gel were mixed and the microbubble concentration was adjusted to approximately 1×10⁹˜2×10⁹ particles/ml (the microbubble liquid).

Component E: chemical, biological and other small molecules or drugs to be used together are prepared. The substance to be used together should be formulated in the desired state of an aqueous solution, an emulsion or a gel, so that the substance is isotonic with human cells with a pH=7.4. For example, chemical, biological or small molecule drugs may be painkillers (such as diclofenac), arbutin, vitamin C phosphate magnesium salt, whitening ingredients (such as nonapeptide-1), gentamycin or glucocorticoid and other applicable substances.

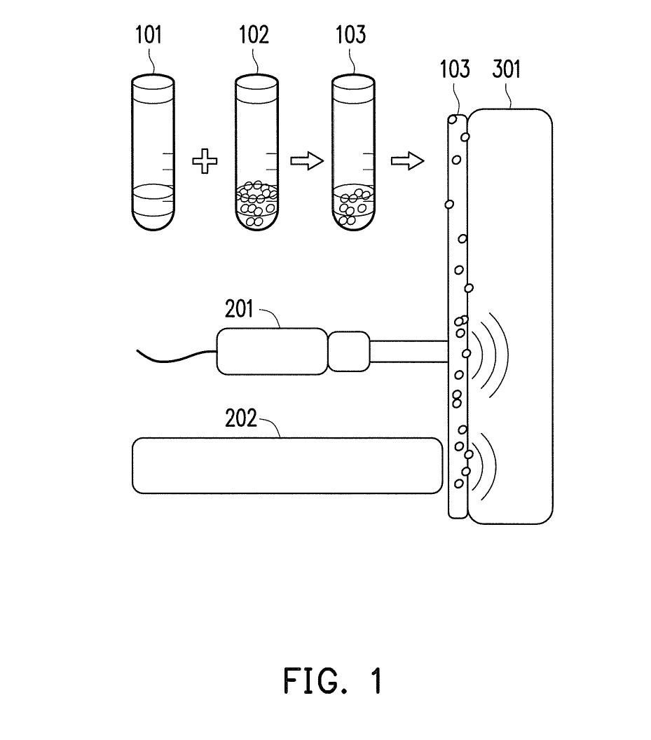

Component F: Component E is used a diluent to dilute Component D 2˜1000 times and the composition obtained after dilution is applied to the surface of the body. Most preferably, Component E is used to dilute Component D 2˜40 times; more preferably, Component E is used to dilute Component D 30˜150 times. Also, Component E is used to dilute Component D 100˜1000 times. According to experimental results, the 10-fold dilution was the best dilution solution applied to the skin surface. Other dilution ratios are effective, and the dilution ratios should be adjusted depending on the application region. In general, in the topical microbubble contrast agent, the concentration of microbubbles ranges preferably from about 2×10⁶˜2×10⁸ particles/mi. In general, the ultrasonic probe directly applied on the outer surface of the living body is in direct contact with Component F for local application of ultrasound with the power of 0.1˜5 W/cm² and the mechanical index (MI) <1.9. In addition, the therapeutic laser beams may be applied to the local region with Component F. To illustrate the principle and design of the present invention, the following embodiments are provided for descriptions. FIG. 1 is a flow chart illustrating the application of the ultrasound microbubble ultrasound contrast agent together with the treatment of ultrasound according to one embodiment of the present invention. First, Component A or Component D (101) is fully mixed with Component B or Component E (102) to obtain Component C or Component F (103), and the resultant Component C or Component F (103) is evenly spread onto the surface of the local region (301). Then the ultrasound probe (201) directly contacts the Component C or Component F (103) spread on the surface of the local region (301), and applying ultrasound (represented by arc lines) in order to enhance the penetration and absorption of the above components or chemicals. The system may further include air gun or laser device (202). The ultrasonic signals of the aqueous or colloid (gel) microbubble ultrasound contrast agent (103), compared with that of water, are pretty significant and have the fundamental frequency and harmonic signals, which keeps various physical effects induced by the ultrasound.

Percutaneous Penetration Experiments

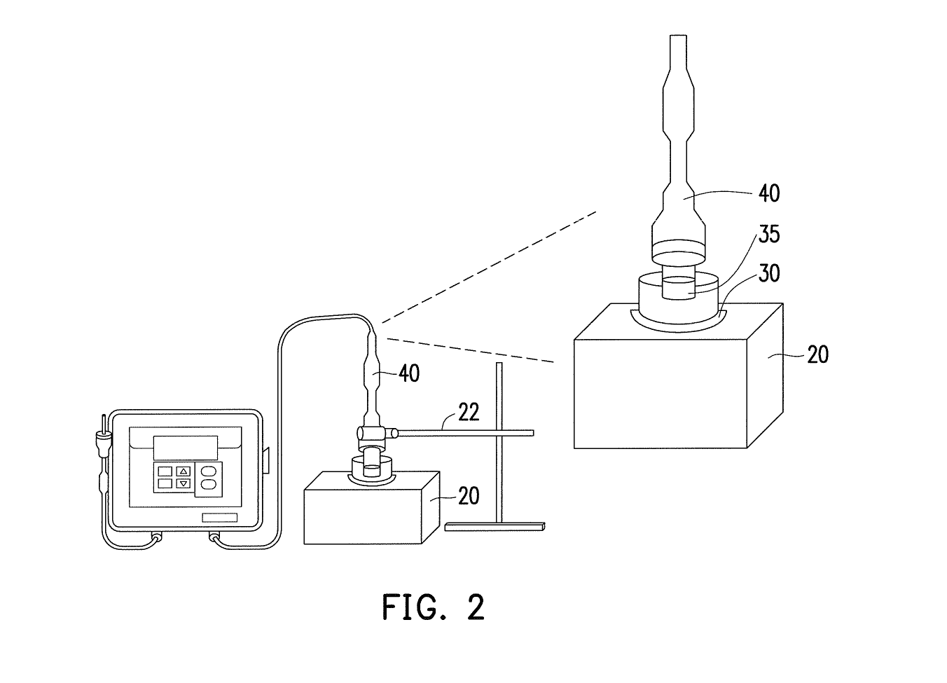

FIG. 2 is a schematic view of a penetration-through experimental system with the tissue simulator according to one embodiment of the present invention. At first, a skin tissue simulator 20 formed of 0.3 wt % agarose gel (agar gel), which simulates the human skin tissue(s), is provided for conducting the penetration-through experiments. The mechanical oscillation wave source may be the ultrasound probe. The ultrasound probe) 40 is mounted on the dropper rack 22 and the ultrasound probe 40 is set at a distance of about 5mm from the tissue stimulator 20. The conductive gel 35 is disposed on the probe 40 so that the conductive gel 35 is located apart from the tissue stimulator 35 with a distance of about 3 mm. See FIG. 2, the perfusion zone 30 is placed above the tissue stimulator 20 and the conductive gel 35 is located outside of the perfusion zone 30. The gel-based microbubble ultrasound contrast agent of this invention is used as the conductive gel 35, and the small molecules or chemicals may be placed in the perfusion zone 30.

Ultrasound applications process: The conductive gel was coated and the ultrasound was applied for 1 minute. The surface of the tissue stimulator is rinsed three times (1000 μl). The control group utilized the saline solution of 0.01 wt % Evans blue dye (0.0001 g Evans blue dye dissolved in 1 ml saline). After the application of the ultrasound, the tissue stimulator was placed in the perfusion zone for 2 to 30 minutes (for example: 5 minutes, 10 minutes, 15 minutes or 20 minutes). After placing in the perfusion zone for a predetermined time (the standing time), the penetration depth of the dye (dye penetration depth) of the tissue stimulator was observed by the microscope and the results were processed by MATLAB program to calculate the dye penetration depth.

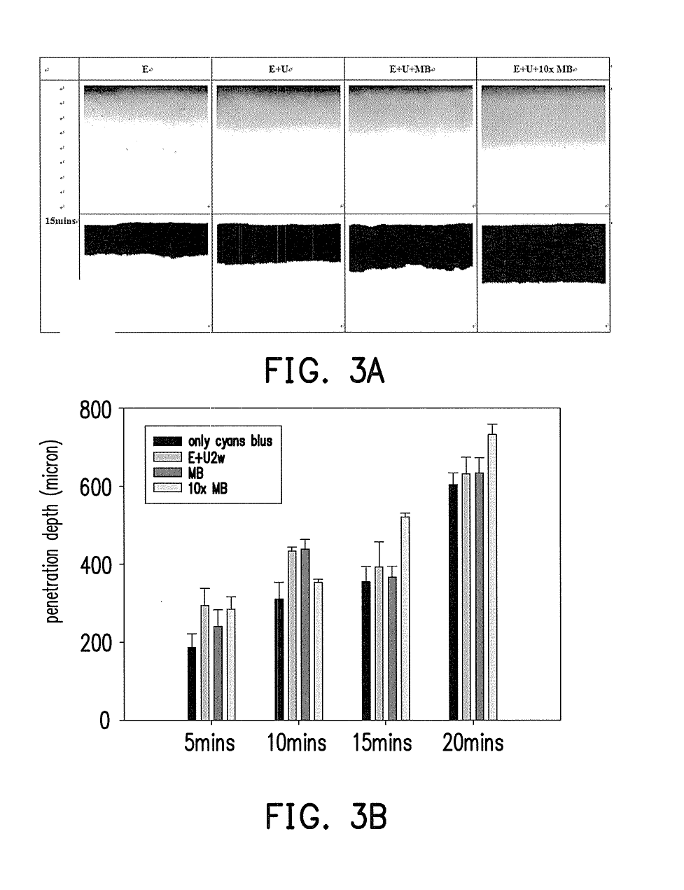

In the following three experiments, different parameters were changed to find the best conditions for the penetration depth of the dye. (1) only Evans blue dye (represented by E); (2) Evan blue dye+ultrasound (represented by E+U); (3) Evans blue dye+ultrasound+microbubble contrast agent (represented by E+U+MB or MB); (4) Evans blue dye+ultrasound+10-fold dilution of microbubble contrast agent (represented by E+U+10×MB or 10×MB); E meant for Evans blue dye; U meant for ultrasound; MB meant for microbubble contrast agents; 10×MB meant for 10-fold dilution of microbubble contrast agent. After the application of the ultrasound and placing in the perfusion zone for a predetermined time, the dye penetration depth was observed by the microscope and the results were processed by MATLAB program to calculate the dye penetration depth. FIG. 3A shows the penetration depth of the agar stimulator in the penetration-through experiments according to one embodiment of the present invention. FIG. 3B is a quantitative diagram showing the relationship of the penetration depth of the agar stimulator in the penetration-through experiments and the standing time according to one embodiment of the present invention.

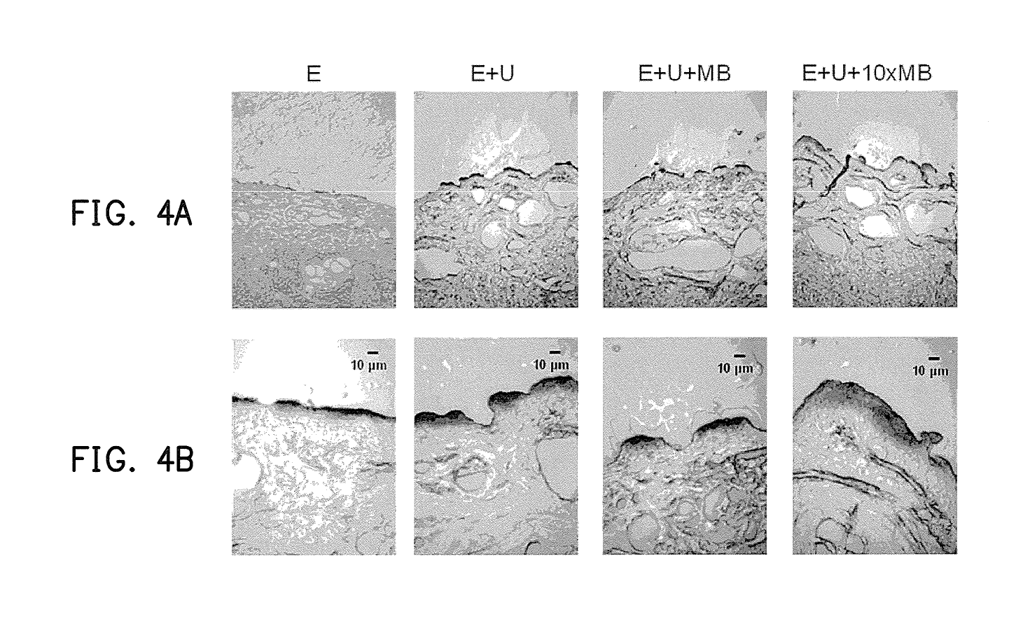

In another experiment, the perfusion zone was placed on the pigskin of 2 mm thickness for conducting the percutaneous penetration experiments, and the experimental system and the methods were similar to the penetration-through experiments of the agar stimulator. The results of the penetration-through experiments are shown in FIGS. 4A-4B. FIG. 4A is a 100-fold magnification showing the percutaneous penetration depth of the penetration-through experiments, while FIG. 4B is a 400-fold magnification showing the percutaneous penetration depth of the penetration-through experiments.

From the experimental results of the penetration-through experiments, the microbubble ultrasound contrast agent of this invention used in combination with the ultrasound can make the dye penetrate deeper or more uniformly. With respect to the agar stimulator, the penetration-through experiments conducted on the pigskin penetration experiments proves that the microbubble ultrasound contrast agent of the present invention do enhance the penetration of small molecules (permeation). During application, it is better to dilute the external use microbubble ultrasound contrast agent of the present invention with a diluent at the dilution ratio of about 1:2 dilution to 1:1000 dilution. The diluent may be the medium itself contained in the microbubble contrast agent of the present invention to increase the proportion of the medium; or the diluent may be a small molecule, a chemical or a medicinal ingredient itself. Further, the medium of the external use microbubble contrast agent is not limited to the traditional liquid state isotonic medium. The microbubbles in the microbubble contrast agent may be made of albumin, polymers, liposomes, copolymers, mixtures or a combination of the aforementioned materials, for example. The microbubble ultrasound contrast agent for topical uses may include the microbubbles in the concentration range of 2×10⁶˜2×10⁸ particleshnl. If a gel medium is used, relatively to the total weight of the composition of the microbubble contrast agent and the medium, the content of the gel medium may be less than or equivalent to 0.2 wt %, which can effectively transfer sound waves. Alternatively, an isotonic saline solution may be used as the medium.

For medical applications, the external use microbubble contrast agent of the present invention may be used in the ear treatments. The microbubble contrast agent of this invention is mixed with the dye and/or one or more medical ingredients and administrated to the inner ear of guinea pigs. The administration of the mixtures may be conducted in different ways to test the delivery efficiency of the dye or the ingredient.

Animal Test Procedures

The animals used in the test are 60 guinea pigs with the normal Preyer's reflex to the sound(s) and are divided into three groups with the following experimental conditions: (1) the tympanic bullae of 24 guinea pigs are filled with the microbubble ultrasound contrast agent mixed the dye indicator and applied with the ultrasound; (2) the tympanic bullae of 9 guinea pigs are filled with the dye indicator and applied with the ultrasound; (3) the microbubble ultrasound contrast agent mixed the dye indicator is applied to the round windows of the remaining 27 guinea pigs, without applying the ultrasound, where the microbubble ultrasound contrast agent mixed the dye indicator is diffused into the round window membrane of the guinea pigs.

In the experiments of the present invention employs Sonoporation Gene Transfection System (ST2000V, NepaGene, Japan), with a probe size of 6 mm and the waveform of square waves. In the experiments, the ultrasound is operated at a frequency of 1 MHz, a duty cycle of 50%, energy of 3 W/cm², is applied for 1 minute. In the experiments, the probe is placed on the body surface facing the round window membrane with a distance of 5 mm.

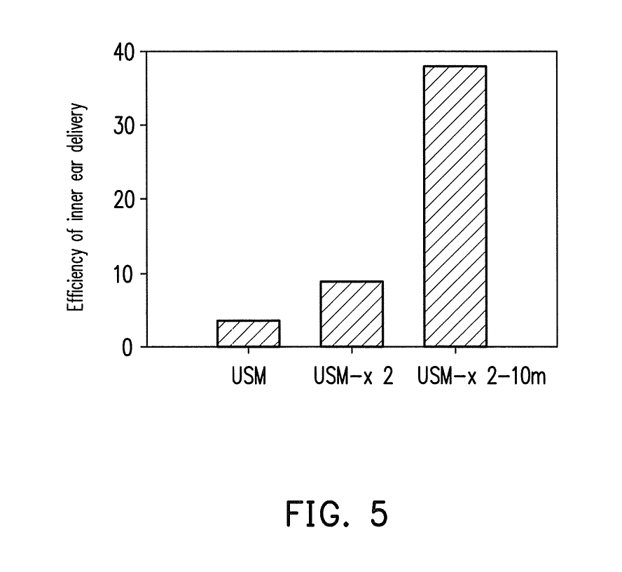

FIG. 5 shows the results of the delivery efficiency using different administration approaches of the microbubble contrast agent in the inner ear treatment experiments. USM refers to give the microbubble ultrasound contrast agent once and apply the ultrasound once, USM×2 refers to give the microbubble ultrasound contrast agent twice and apply the ultrasound twice, USM×2-10 m refers to give the microbubble ultrasound contrast agent twice and apply the ultrasound twice and stranded for 10 minutes. Compared to the control group of delivering the dye or drug into the inner ear through the diffusion effect, the experimental results indicate that the ultrasound used together with the microbubble ultrasound contrast agent can enhance the drug delivery efficiency. That is, the delivery efficiency of the administration approaches USM, USM×2, USM×2-10 m is respectively 3.5 times, 8.8 times, 37.9 times of that of the control group. In addition, in order to deliver gentamycin into the inner ear, the microbubbles ultrasound contrast agent of this invention is used along with the application of the ultrasound. By using such approach, the concentration of gentamycin delivered into the cochlear tissues is significantly higher than that of the control group without applying the ultrasound. Hence it is confirmed that the microbubble contrast agent can enhance the delivery of the chemical and promote the absorption of the drug or small molecules.

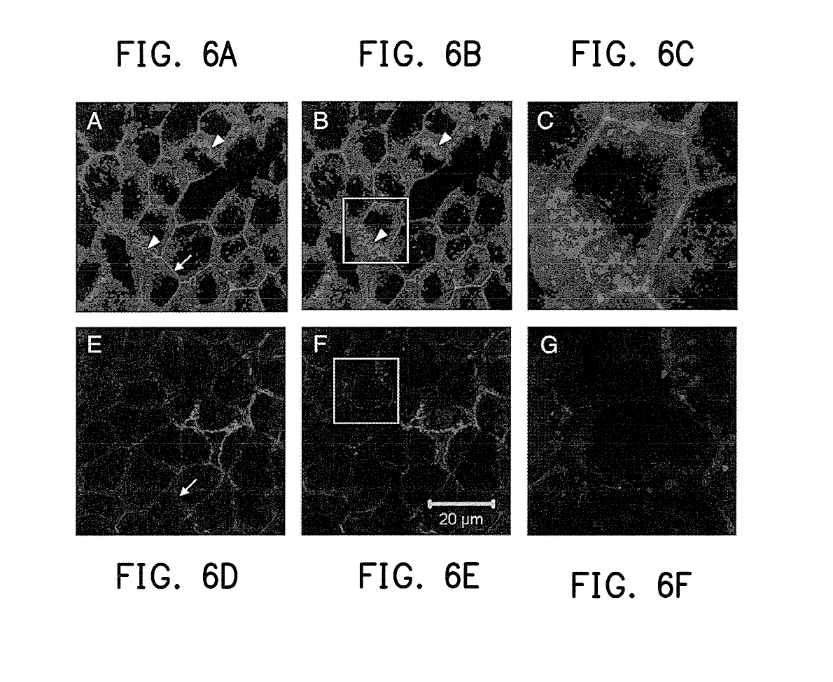

FIGS. 6A˜6F show the delivery results of the green dye indicator entering into the round window membrane cells of the inner ear under different administration approaches. FIGS. 6A˜6C show the delivery results of the experimental groups using the ultrasound microbubble contrast agent mixed with the green dye indicator and operated with the ultrasound. FIGS. 6D˜6F show the delivery results of the control groups using the ultrasound microbubble contrast agent mixed with the green dye indicator but without applying the ultrasound (through the diffusion effect). Compared the results of little or no green dye entering into the round window membrane cells in FIGS. 6D˜6F, the results of FIGS. 6A˜6C show much more green dyes entering into the round window membrane cells.

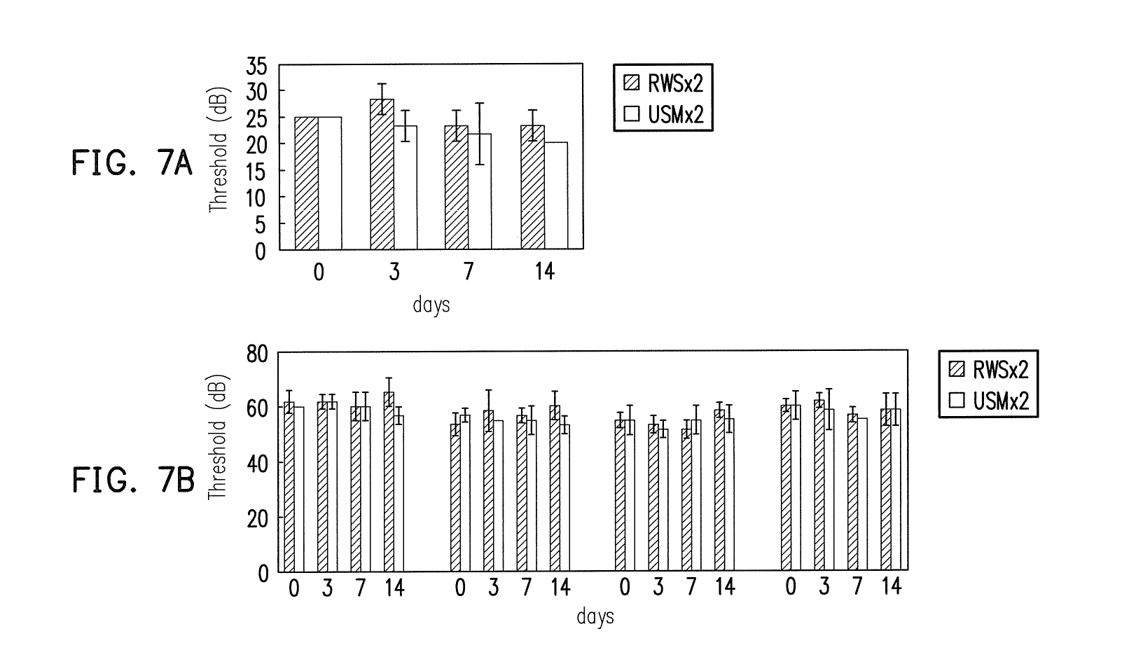

In addition, in order to verify whether the microbubble ultrasound contrast agent(s) of the present invention will do harm to the cells in the inner ear cochlea, the present invention also perform hearing threshold functional evaluation experiments on the guinea pigs experiencing the aforementioned animal tests. FIGS. 7A˜7B show the results of the auditory brainstem response tests of the animals following the animal tests. The animals in the experimental group administrated with the drug and ultrasound (denoted as USM) or in the control group administrated with the drugs without ultrasound (denoted as RWS) further went through the auditory brainstem response tests on ticking sounds (FIG. 7A) and plosive sounds (FIG. 7B). The results show no difference between two groups in the hearing thresholds, indicating that the microbubble ultrasound contrast agents acting on the inner ear cochlea causes no harm to the cells in the auditory system.

The ultrasound applicable in the present invention is preferably a non-focusing type low-energy ultrasound, and its energy range is of the MI=0.2˜0.4, compared to the FDA provisions for the medical ultrasound being below the MI of 1.9 or the ultrasound for ophthalmic uses being below the MI of 0.2, the energy range of the ultrasound applicable in the present invention is far below these ranges. Furthermore, the energy range of the ultrasound used in the present invention does not cause local temperature variations. In the experiments of the present invention, it is found that the temperature difference is only plus or minus 0.1 degree during the operation. Therefore, the energy range of the ultrasound used in the present invention will not have thermal effects.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. An external use microbubble ultrasound contrast agent, comprising:
a medium, wherein the medium is in a form of an aqueous solution or a gel form; and
a plurality of microbubbles dispersed in the medium, wherein a concentration of the microbubbles ranges from 1×10⁹ to 2×10⁹ particles/ml.

2. The microbubble ultrasound contrast agent as claimed in claim 1, wherein a material of the microbubbles is selected from albumin, polymers, liposomes, copolymers or mixtures thereof or a combination of thereof, and the medium is selected from an isotonic saline solution, an agar gel, an aloe gel, a topical gel or a combination of thereof.

3. The microbubble ultrasound contrast agent as claimed in claim 1, wherein the medium is a gel form medium and a content of the gel form medium is less than or equivalent to 0.2 percentages by weight of a total weight of the microbubble ultrasound contrast agent.

4. The microbubble ultrasound contrast agent as claimed in claim 1, wherein a particle size of the microbubbles ranges from 0.5 micrometers to 2.5 micrometers.

5. The microbubble ultrasound contrast agent as claimed in claim 1, further comprising a chemical or small molecules, wherein the chemical or the small molecules are percutaneously absorbed by a biological body.

6. A method of enhancing percutaneous absorption of a chemical or small molecues through a topical region of a biological body surface, comprising:
applying a microbubble ultrasound contrast agent to the topical region of the biological body surface, wherein microbubble ultrasound contrast agent comprises a medium and a plurality of microbubbles dispersed in the medium, the medium is in a form of an aqueous solution or a gel form, and a material of the microbubbles is selected from albumin, polymers, liposomes, copolymers or mixtures thereof or a combination of thereof;
applying the chemical or the small molecules to the topical region; and
applying a mechanical oscillation wave source to be in direct contact with the topical region applied with the microbubble ultrasound contrast agent and the chemical or the small molecules, through mechanical waves generated by the mechanical oscillating energy source acting on the microbubbles, so as to increase the percutaneous absorption of the chemical or the small molecules.

7. The method of claim 6, wherein a concentration of the microbubbles ranges from 2×10⁶ to 2×10⁸ particles/ml, relative to the total volume of the microbubble ultrasound contrast agent and the chemical or the small molecules.

8. The method of claim 7, further comprising using the chemical or the small molecules as a diluent to dilute the microbubble ultrasound contrast agent 2-1000 times.

9. The method of claim 6, wherein the steps of applying the microbubble ultrasound contrast agent and applying the chemical or the small molecules are performed separately.

10. The method of claim 6, wherein a particle size of the microbubbles ranges from 0.5 micrometers to 2.5 micrometers.

11. The method of claim 6, wherein the mechanical oscillation wave source includes an ultrasound source and/or a laser source.
